(19) United States
(12) Patent Application Publication
(10) Pub. No.: US 2026/0355500 A1
(43) Pub. Date: Nov. 12, 2026

(54) Analysis of Rhythm Regulation for Guiding Improved Synchronization with Pump Function of the Heart

(71) Applicant: Lifebridge Technologies LLC, Dayton, OH (US)

(72) Inventor: Mark P. Anstadt, Kettering, OH (US)

(21) Appl. No.: 19/740,226

(22) Filed: Jul. 13, 2026

Related U.S. Application Data
(63) Continuation-in-part of application No. 17/825,343, filed on May 26, 2022, now Pat. No. 12,697,482.
Application No. 17/825,343 is a continuation-in-part of application No. 17/208,776, filed on Mar. 22, 2021, now Pat. No. 11,383,076.
(60) Provisional application No. 63/086,478, filed on Oct. 1, 2020.

(51) Int. Cl. A61M 60/289

(52) U.S. Cl. CPC A61M 60/289

(57) ABSTRACT

A control system and method for a cardiac assist device. A cardiac assist device selectively applies mechanical forces to the patient's heart. The heart is monitored to determine time intervals between cardiac cycles. Stability for the rhythm is determined. The cardiac assist device is dynamically controlled as a function of the stability. The cardiac assist device applies the mechanical forces to the patient's heart to cause said stability to approach regularity. Regularity is a measure of the consistency of the time intervals between cardiac cycles with the perfect regularity being a 1:1 ratio between the current time interval and the prior time interval(s).

### RELATED APPLICATIONS

This application is a continuation-in-part of U.S. patent application Ser. No. 17/825,343, filed May 26, 2022, which is a continuation-in-part of U.S. patent application Ser. No. 17/208,776 filed Mar. 22, 2021, now U.S. Pat. No. 11,383,076, which claims the benefit of U.S. Provisional Patent Application No. 63/086,478, filed Oct. 1, 2020, the entirety of each of which are herein incorporated by reference.

### BACKGROUND OF THE INVENTION

### 1. Field of the Invention

In general, the present invention relates to auxiliary cardiac assist devices that directly contact the heart and assist the heart in pumping blood. More particularly, the present invention relates to control systems that activate the auxiliary cardiac assist devices and attempt to control the operation of the auxiliary cardiac assist devices in synchronization with some aspect of the cardiac cycle to better assist in the functionality of the heart.

### 2. Prior Art Description

There are medical circumstances when it is beneficial to attach a cardiac assist device to, in, or near, an injured or failing heart. The cardiac assist device supplements the pumping action of the heart by imparting forces directly onto one or more surfaces of the heart in an effort to artificially maintain the physiologic function of the heart. Most of these cardiac assist devices mechanically compress the heart to aid systolic pump function and/or mechanically expand the heart to aid in diastolic pump function.

The heart is a complex organ. When the heart pumps, various muscle tissue within the heart contracts in an extremely specific pattern. During each cardiac cycle, a healthy heart has an orderly progression of depolarization that starts with pacemaker cells in the sinoatrial node. The progression spreads throughout the atrium and passes through the atrioventricular node into the bundle of His and into the Purkinje fibers. Lastly, the progression spreads throughout the ventricles. The electrical impulses created by the progression can be detected using electrocardiogram equipment. The electrical impulses created by the progression create a corresponding pattern of contractions within the heart. When the ventricles of the heart contract, the contracting cells have a unique pattern. This pattern of contraction is termed mechanical cardiac synchrony. The mechanical cardiac synchrony produces a coordinated mechanical expulsion of blood from the ventricles. There also is a mechanical synchrony for the progression of diastolic contractions during the filling of the ventricles. A normal, healthy heart exhibits normal mechanical cardiac synchrony. A diseased, damaged, or failing heart often does not have normal mechanical cardiac synchrony and is referred to as being dyssynchronous.

There are three main electrical components contained within the cardiac cycle waveform that are recorded in an electrocardiogram. These three components create corresponding mechanical contractions in the tissues of the heart. The three main electrical components include the P-wave, the QRS complex, and the T-wave. The P-wave represents depolarization of the atria, wherein atrial depolarization spreads from the SA node towards the AV node, and from the right atrium to the left atrium. This process typically takes about 80 milliseconds. The QRS complex, which represents depolarization of the ventricles, typically takes between 80 milliseconds and 100 milliseconds. The ventricles have more muscle mass than the atria. Accordingly, the QRS complex usually has a larger amplitude than the P-wave. Since the QRS complex corresponds to ventricular contraction and is a prominent signal. The QRS complex contains the R-wave, which is often used to determine the “time” of contraction as the heart beats. Finally, the T-wave represents the repolarization of the ventricles. This process typically takes about 160 milliseconds. Accordingly, it will be understood that a heartbeat is a progression of complex contractions of the heart. It is this complex progression of contractions that is often adversely affected when the heart becomes diseased or damaged. A damaged or diseased heart contracts with “dysynchrony” and pumps blood in an inefficient manner.

There are many cardiac assist devices in the prior art that can apply forces to the exterior of the heart. Some devices, such as U.S. Pat. No. 7,494,459, to Anstadt and U.S. Pat. No. 6,076,013 to Brennan, disclose cup shaped devices that encircle most of the ventricles. Other devices, such as U.S. Patent Application Publication No. 2015/0080640 to Lillehei and U.S. Patent Application Publication No. 2007/0197859 to Schaer et al., disclose bands that surround only a portion of the ventricles. One of the problems associated with such prior art cardiac assist devices is that the devices are mechanisms that expand and/or contract when instructed. Accordingly, the operation of such devices tends to be binary, that is on/off. It is therefore very difficult to control a cardiac assist device so that the cardiac assist device accurately assists a heart in achieving the complex mechanical synchrony of a healthy heart. Most often, the result is a compromise where hemodynamic feedback is used to control the cardiac assist device. Such control of the cardiac assist device can be used to mimic some of the complex contractions of the heart, but not all. The result is that the cardiac assist device may fight the natural contractions of the heart in certain places at certain times. This can further fatigue some heart tissue and make the heart pump less effective. Furthermore, by being out of synchronization with the heart, the cardiac assist device can hinder the heart in recovering a normal stable rhythm. When a heart is recovering, it is not unusual for the heart to beat faster than normal. If a cardiac assist device is set to a different pace, the cardiac assist device can be completely out of synchronization with the heart's contractions. This can hinder and even prevent the heart from recovering to a point where it can beat without external assistance.

A need therefore exists for an improved control system for synchronizing a cardiac assist device to the heart. In this manner, the cardiac assist device can apply appropriate forces to the heart in a manner that assists a diseased or damaged heart to both achieve better pacing synchrony and to achieve better mechanical cardiac synchrony. This need is met by the present invention as described and claimed below.

### SUMMARY OF THE INVENTION

The present invention is a system and method for arriving at device synchronization between the forces applied by a cardiac assist device and the natural rhyme of a heart. The system and method require the use of only one biometric signal that is indicative of the heart's pump function in order to guide and adjust the cardiac assist device. The operational rate of the cardiac assist device is changed in response to increased irregularity of the measured biometric signal(s). The regularity of the biometric signal(s) are used as a means to arrive at device synchrony. The biometric signals can be any signal indicative of the heart's rhythm, such as an EKG, an arterial pressure signal or a measure of cardiac strain peaks. Methodical tools exist in physics, engineering, and audio signal processing to auto correlate time shifted signals to identify hidden periodicities. For rhythmic signals, a measure in seconds is between 1 and an inter onset interval (IOI). Evaluating the mathematical consistency of these intervals using variance, ratio-based spectra or entropy can be used to assess rhythmic regularity and temporal patterning of a sequence. To ensure a metronome signal is regular, one can calculate the coefficient of variation. The coefficient of variation is the standard deviation or amount of fluctuation in the interval length. Autocorrelation or signal processing measures the similarity of the signal against itself over varying time lags. Fourier analysis divides time into uniform intervals. The Fourier transform deconstructs complex rhythmic waveform into underlying periodic frequencies. Thus, a single biometric signal can be used as a means of measuring the regularity of the heart's function (e.g., arterial pulse pressure, Strain, flow, ECG, etcetera). This measure of regularity is then used to adjust the rate of the drive system acting on the heart. The adjustment of the rate of the drive system in response rhythm irregularity guides the drive toward synchronous support with the cardiac assist device and the underlying heart function. In general, the device rate is gradually increased when an irregular rhythm threshold is detected. A new setting is assessed to determine if regularity is improved. The ongoing process allows constant adjustment of the device rate to achieve ideal synchrony with the functioning heart and is particularly important for during periods of recovery.

Accordingly, the present invention is a control system for a cardiac assist device and the method of supporting the functionality of a heart with the cardiac assist device. A cardiac assist device is positioned to affect the patient's heart, wherein the cardiac assist device selectively applies mechanical forces to the patient's heart. The patient's heart is monitored to determine time intervals between cardiac cycles, wherein the time intervals include a current time interval and a prior time interval.

Stability for the rhythm is determined, wherein the stability is indicative of time regularity within the rhythm. The cardiac assist device is dynamically controlling as a function of the stability. The cardiac assist device applies the mechanical forces to the patient's heart to cause the stability to approach regularity. Regularity is a measure of the consistency of the time intervals between cardiac cycles with the perfect regularity being a 1:1 ratio between the current time interval and the prior time interval.

### BRIEF DESCRIPTION OF THE DRAWINGS

For a better understanding of the present invention, reference is made to the following description of exemplary embodiments thereof, considered in conjunction with the accompanying drawings, in which:

FIG. 1 is an exemplary schematic overview of the present invention system shown in conjunction with a heart;

FIG. 2 shows the exemplary embodiment of FIG. 1 engaging the heart;

FIG. 3 is a graph showing an exemplary ventricular strain profile produced by the cardiac assist device of the present invention system;

FIG. 4 shows an alternate embodiment for the cardiac assist device, that has a different area of contact than that of the exemplary embodiment of FIG. 1 and FIG. 2;

FIG. 5 shows the cardiac assist device in engagement with a heart to illustrate surface area of contact and areas of displacement;

FIG. 6 is a graph that plots surface area of contact with pressures needed to be generated by the cardiac assist device;

FIG. 7 shows a cardiac waveform and illustrates wave intervals that can be used to determine cardiac cycle rate;

FIG. 8 is a flow diagram that illustrates an exemplary operating methodology; and

FIG. 9 is a graph depicting the cycles featuring loss of ventricular capture.

### DETAILED DESCRIPTION OF THE DRAWINGS

Although the present invention system and methodology can be embodied in many ways, only two exemplary embodiments are illustrated. Exemplary embodiments are being shown for the purposes of explanation and description. The exemplary embodiments are selected in order to set forth some of the best modes contemplated for the invention. The illustrated embodiments, however, are merely exemplary and should not be considered as limitations when interpreting the scope of the appended claims.

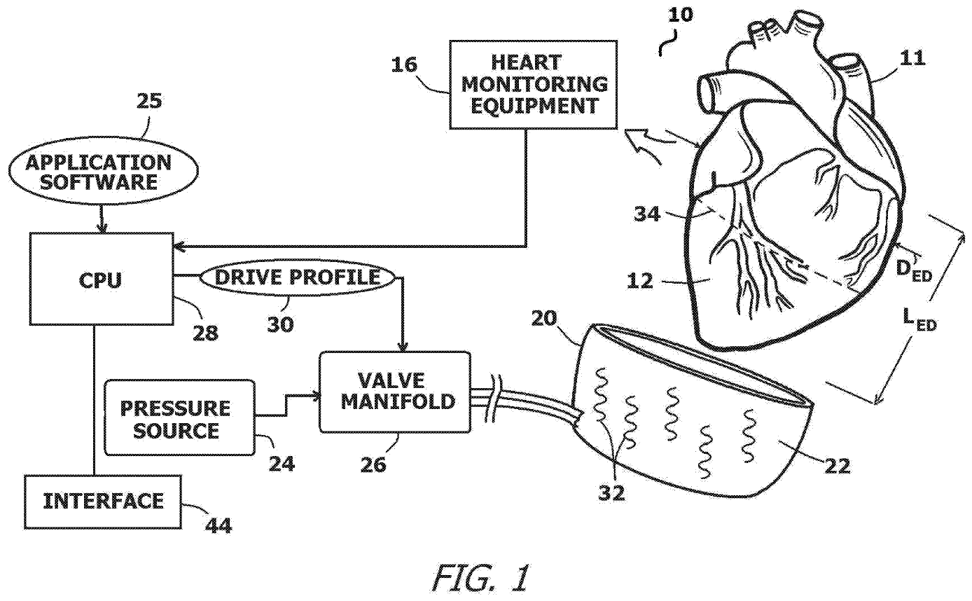

Referring to FIG. 1, an exemplary embodiment illustrating the present invention system 10 is shown. In the illustrated embodiment, a heart 11 is shown for better understanding of context. Every individual has a different physiology. The heart 11 has measurable dimensions that are unique for a particular person. One of the measurable dimensions is the maximum diameter D_{(ED)}. The maximum diameter D_{(ED)} corresponds to the diameter of the myocardium at its widest point as measured at the end of the diastolic cycle. Likewise, the heart 11 has a measurable length L_{(ED)} that corresponds to the length of the ventricles at the end of the diastolic cycle. These dimensions of the heart 11 can be readily obtained from various medical scanning equipment, such as x-rays, ultrasounds, MRIs, and the like. Furthermore, it is understood that the heart 11 pumps to a cardiac cycle, wherein the heart 11 contracts with a regular contraction rhythm. Although the contraction rhythm can be irregular, it has a general average rate of contraction over increments of time. The contraction rhythm of the heart 11 can also be quantified using heart monitoring equipment 16. The heart monitoring equipment 16 can be any system that detects and measures a biometric signal indicative of pulse rate. Such heart monitoring equipment 16 includes, but is not limited to pulse monitors, blood pressure monitors, ECG units, flow rate monitors, x-ray scanners, and ultrasonic scanners.

The measured maximum diameter D_{(ED)} and measured length L_{(ED)} of the heart 11 can be used to accurately calculate other characteristics of the heart 11. For instance, the measured maximum diameter D_{(ED)} correlates directly to the volume V_{(ED)} within the ventricles 12 at the end of the diastolic cycle. The relationship between the measured maximum diameter D_{(ED)} and the measured volume V_{(ED)} is explained in detail in U.S. Pat. No. 11,383,076, the disclosure of which is herein incorporated by reference.

A cardiac assist device 20 is provided. There are multiple cardiac assist devices 20 in the prior art and in the commercial marketplace, many of which can be adapted for use with the present invention system 10. The cardiac assist device 20 illustrated is indicative of those known and available. Some cardiac assist devices are positioned in or near the heart and apply mechanical forces to heart in the area of contact. In the illustrated embodiment, the cardiac assist device 20 contains a cup or band 22 that surrounds the ventricles 12 of the heart 11 in part or in whole. Depending upon the make and model of the cardiac assist device 20 selected, the band 22 expands and/or contracts, therein mechanically assisting the pumping action of the heart 10. The cardiac assist device 20 contains various internal tubes and compartments that expand or contract depending upon the pressure supplied to those tubes and compartments. The pressure can be pneumatically or hydraulically supplied from an external pressure source 24, such as a hydraulic pump or air compressor. The pressure supplied to the various tubes and compartment is controlled by a valve manifold 26. The operation of the valve manifold 26 is controlled by application software 25 running in a central processing unit 28. The application software 25 produces drive profiles 30 that control the valve manifold 26. The drive profiles 30 cause the valve manifold 26 to regulate the pressures in the cardiac assist device 20, wherein the cardiac assist device 20 applies forces to the heart 11 that reflect the drive profiles 30. The present invention system 10 creates feedback between the heart 11 and the central processing unit 28, so that the drive profiles 30 controlling the operations of the cardiac assist device 20 can better synchronize with the natural contractions of the heart 11.

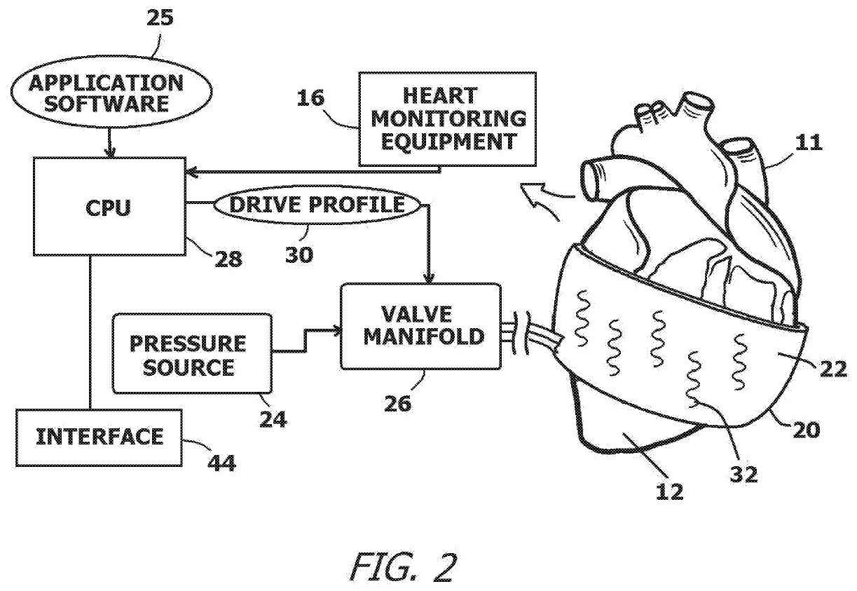

The pumping characteristics of the heart 11 can be expressed using multiple biometric signals. Data from the biometric signals are used to extrapolate the drive profiles 30 that are utilized by the central processing unit 28 to operate the cardiac assist device 20. As can be seen in FIG. 2, the cardiac assist device 20 is placed around the ventricles 12 of the heart 11. At least one biometric signal that is indicative of pulse rate is directly measured. Some biometric signals, such as ECG signals are directly indicative of pulse rate. Others, such as heart strain profiles, are processed to obtain the data needed. Heart strain profiles can be accomplished passively, using data from X-ray scans, MRI scans or other scans that image the heart and detect strains. Alternatively, the heart strain profiles can be actively obtained by embedding strain gauges into the cardiac assist device. Regardless of the technique used, any strain ε(t) experienced by the heart 11 can be detected. When the effects of the cardiac assist device 20 are ignored, the strain ε(t) is created by the inherent function of the heart 11. This assumes the heart 11 is not completely arrested and is capable of providing some pumping function. Should the heart 11 be fully arrested, the strain ε(t) would solely represent the function imparted by the cardiac assist device 20. The goal of the overall system 10 is to regulate the cardiac assist device 20 so that the forces applied by the cardiac assist device 20, combined with the remaining inherent functionality of the heart, create the optimal strain/strain rate profiles for the heart 11.

In FIG. 1, a band line 34 is shown that represents the ventricular epicardium on the heart 11. To understand strain ε(t) in the heart 11, it should be imagined that the band line 34 is opened and flattened into a straight line. The band line 34 has a length L1. When the heart 11 contracts, that band line 34 has a shorter length L2. In the heart 11, strain ε(t) is the change in length (ΔL) over the initial length (L1). Since the heart 11 can be considered as containing many stacked layers, both above and below the ventricular epicardium, strain ε(t) varies along the length and is therefore a function of length.

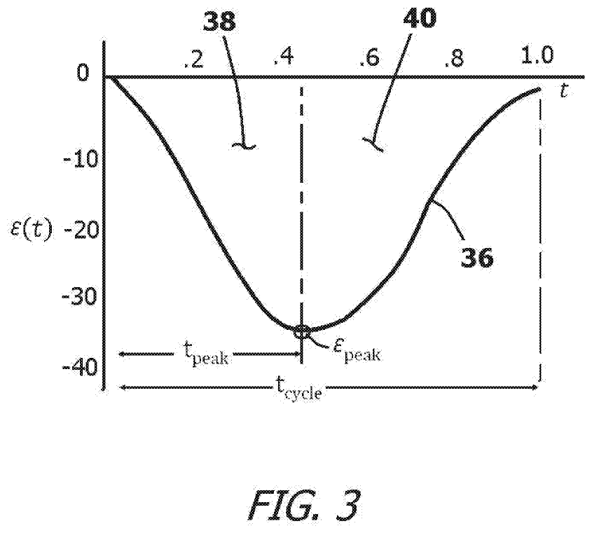

The cardiac assist device 20 is controlled by drive profiles 30 in a manner that alters the ventricular strain profiles of the heart 11 in a complementary manner. Referring to FIG. 3, an optimal ventricular strain profile 36 for a healthy heart is shown, wherein the heart is average for the physiology of the patient. Such an optimal ventricular strain profile 36 can be generated for any heart of a given maximum diameter D_{(ED)}. As can be seen, the strain ε(t) has a compression phase 38 and a retraction phase 40 over time (t). As can also be seen, there is a peak strain ε₍ₚₑₐₖ₎, a time to peak strain t (peak), and an overall cycle period t(cycle).

An estimation for the strain ε(t) in the compression phase 38 can be estimated using the following equation:

$$ϵ ( t ) = \frac{{ε}_{peak}}{2} ( sin ( \frac{π}{{t}_{peak}} \times t - \frac{3}{2} π ) - 1 ) \qquad Equation 1$$

An estimation for the strain ε(t) in the retraction phase 40 can be estimated using the following equation:

$$ϵ ( t ) = \frac{{ϵ}_{peak}}{2} ( sin ( \frac{π}{{t}_{cycle} - {t}_{cycle}} \times ( t - {t}_{peak} ) - \frac{π}{2} ) - 1 ) \qquad Equation 2$$

The value for ε₍ₚₑₐₖ₎ can be estimated from the measured maximum diameter D_{(ED)} of the heart 11 using the following equation:

ε₍ₚₑₐₖ₎≈−0.1254(D_{(ED)})−7.7135 Equation 3

The value for t_{(cycle)} is determined by the following equation.

t_{(cycle)}=(Activation Rate/60)⁻¹ Equation 4

The activation rate can be determined from the measured maximum diameter D_{(ED)} using the following equation.

Activation Rate≈−1.2089 D_{(ED)}+221.46 Equation 5

From the above description and equations, it will be understood that an optimal ventricular strain profile 36 for healthy hearts of different physiologies can be estimated. What remains is the problem of how to actively control the cardiac control device 20 so that it applies the needed strains to a damaged or diseased heart so that the heart can better function near its optimal strain profile.

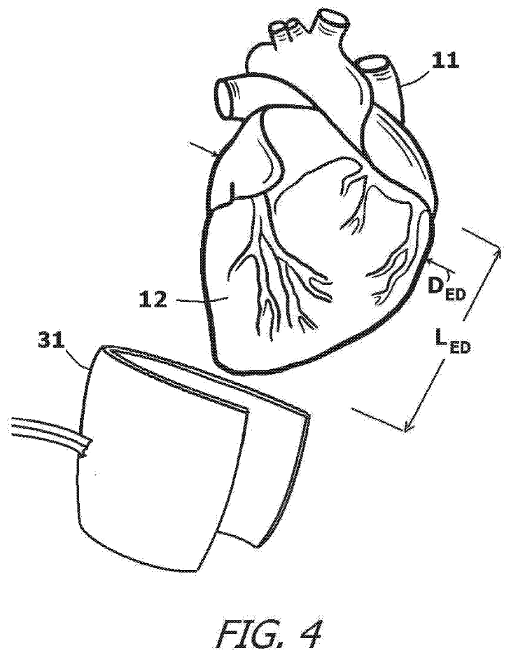

In FIG. 2, the exemplary cardiac control device 20 has a band that encircles the heart. Referring to FIG. 4, an alternate embodiment is shown, wherein the cardiac assist device 31 does not encircle the heart 11 but contacts areas of the heart 11. Depending upon the size of the heart 11 and the make and model of the cardiac assist device, there will be different areas of contact onto which forces are applied to the heart 11.

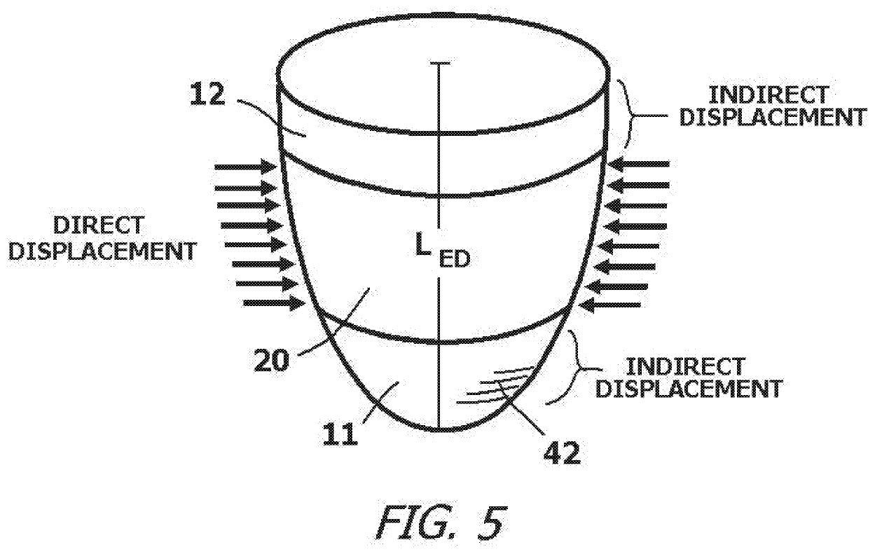

Referring to FIG. 5 in conjunction with FIG. 2, it will be understood that the exemplary cardiac assist device 20 is applied to the heart 11 so that the cardiac assist device 20 covers all, or part, of the ventricles 12. Accordingly, the surface area on the heart 11 that the cardiac assist device 20 engages is very important in determining how much strain the cardiac assist device 20 must supply. The force that the cardiac assist device 20 applies to the heart 11 is divided by the surface area of contact. As such, a cardiac assist device 20 that covers only part of the ventricles 12 must apply more force than one that covers most all of the ventricles 12, since force is inversely proportional to surface area.

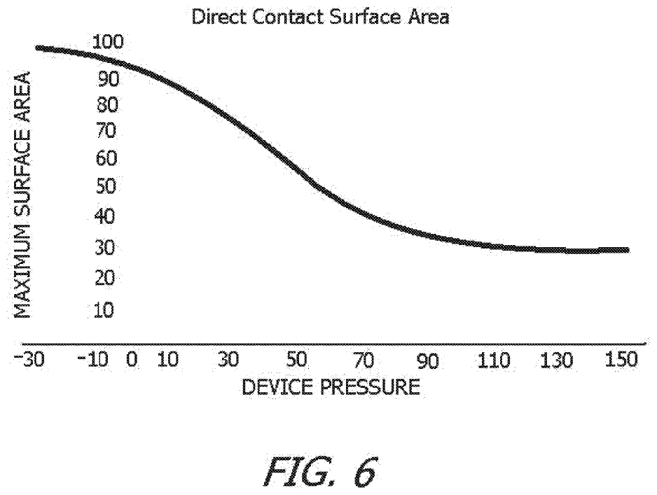

Furthermore, if the cardiac assist device 20 covers only part of the heart 11, as is shown, the cardiac assist device 20 creates both direct displacement and indirect displacement of the heart tissue. Direct displacement is created by the forces applied directly to the heart 11 by the cardiac assist device 20. These direct forces compress the heart 11. This direct compression, in turn, causes displacement in adjacent heart tissue that is not in contact with the cardiac assist device 20. This indirect displacement also alters the shape and volume of the heart 11. These changes can cause a change in the rhythm of the cardiac cycle. To further complicate matters, the direct displacement and the indirect displacement created by the cardiac assist device 20 combine to elongate the heart 11. That is, the displacements provided by the cardiac assist device 20 make the measured length L_{(ED)} longer. As the heart elongates, a smaller percentage of the heart 11 remain in direct contact with the cardiac assist device 20. This reduces contact surface area. This, in turn, requires that the contact surface area supply greater forces to maintain the same compression forces on the heart 11. The changes in the pressures that the cardiac assist device 20 must provide as a function of surface area are shown in the graph of FIG. 6.

Referring to FIG. 5 in conjunction with FIG. 6 and FIG. 2, it will be understood that as the heart 11 elongates, changes in the volume of the heart 11 can be estimated by imagining the heart as a series of stacked disks 42 that number 1 to (n). Some of those stacked disks 42 are in the area of direct displacement. Other of the stacked disks 42 are in areas of indirect displacement. The volume of each disk 42 in the area of direct displacement can be estimated using the following equation.

$${V}_{Disk} = {π ( {r}_{0} ( 1 + \frac{Strain}{100} ) )}^{2} h \qquad Equation 6$$

The variable “V_{disk}” is the volume of the disk. The variable “R” is the original radius of the disk. The variable “H” is the height selected for the disk, which is the length increment between disks. The variable “n” is the number of disks.

The volume of the disks 42 in the areas of indirect displacement can be estimated using the following equation.

V_{Disk}=π(r₀)²h Equation 7

The total volume of displacement can therefore be estimated using the following equation.

$${V}_{total} = {{\sum }_{i = 1}}^{n} {V}_{{Disk}_{n}} \qquad Equation 8$$

Accordingly, the changes in volume of the heart 11 created by the application of compression forces by the cardiac assist device 20 can be estimated. Changes in volume correspond to changes in heart length, which correspond to changes in surface area and pressure requirements via the graph of FIG. 6.

Returning to FIG. 1 and FIG. 2, it will be understood that the central processing unit 28 can directly monitor strains and strain profiles in real time. If ventricular strain is measured using strain scan data from a heart imaging machine, then that strain scan data is fed to the central processing unit 28. Furthermore, the central processing unit 28 can receive signals from one or more other pieces of heart monitoring equipment 16. Such heart monitoring equipment 16 can monitor cardiac cycle rate as a function of blood flow rate, arterial pressure, electrocardiogram, peak strain, or the like. In this manner, the central processing unit 28 can dynamically monitor heart rate.

A user interface 44 is provided, wherein medical personnel can enter the type of cardiac assist device 20 being used and the heart physiology of the patient. Given heart physiology and the make and model of the cardiac assist device 20, the contact area between the heart 11 and the cardiac assist device 20 of the heart 11 can be readily determined. The contact area corresponds to the area of direct displacement that will be affected by the cardiac assist device 20. Given heart physiology and contact area, the area of indirect displacement can also be determined.

The central processing unit 28 runs application software 25 that synchronizes the operation of the cardiac assist device 20. In a first process, a pacing synchronization is achieved where the contraction of the cardiac assist device 20 corresponds to the rhythm of the heart. The “rhythm” of the heart is measured as the time interval between the same waveform elements in sequential cardiac cycles. This is typically the RR interval in an ECG signal. In a second process, the forces applied by the cardiac assist device are dynamically adjusted to achieve proper mechanical cardiac synchrony. That is, the applied forces improve ventricular function by encouraging the ventricular muscles to contract in a more synchronous manner and alter the cardiac contractions from a dyssynchronous pattern to a more favorably pattern.

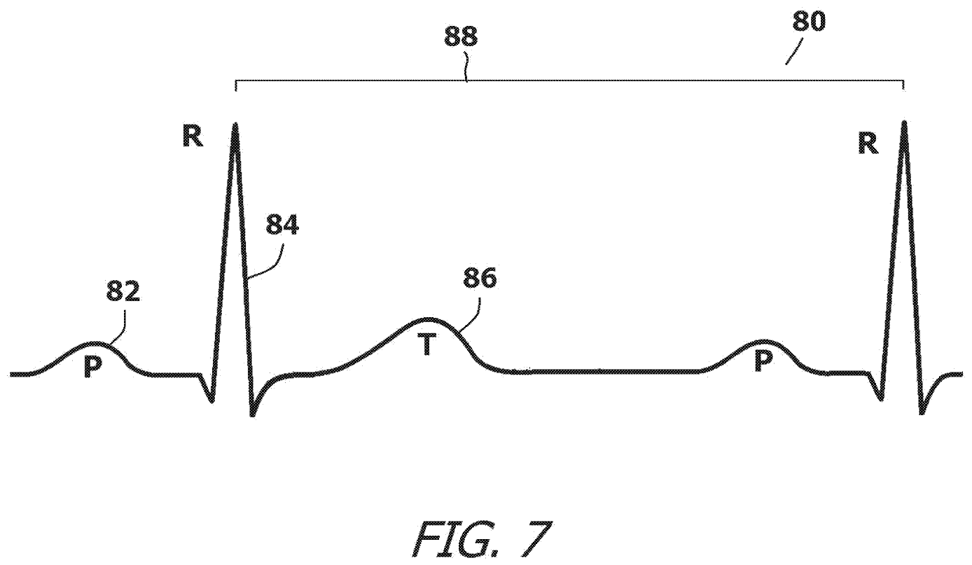

Referring to FIG. 7 an exemplary ECG waveform 80 is shown to identify the time interval being measured. Each cardiac pumping cycle shown on the ECG waveform 80 has a prominent P-wave 82, an R-wave 84, and a T-wave 86. The P-wave 82 represents the electrical depolarization (activation) of the atria, which initiates atrial contraction. The R-wave 84 is the first upward or positive deflection in the QRS complex of an electrocardiogram (ECG). The P-wave 82 represents the electrical impulse as it stimulates the ventricular walls and causes contraction. The T-wave 86 represents ventricular repolarization. A hearts “rhythm” is the time interval between matched wave points in the cardiac cycle. This can be measured in a few ways. The most common measure of the time interval between beats is the RR interval 88. The RR interval 88 is the time elapsed between two successive R-wave peaks. The RR interval 88 is the standard metric used to calculate heart rate. Other intervals, such as the PP interval and the TT interval can also be used, wherein the PP interval is the time between P-wave peaks and the TT interval is the time between T-wave peaks.

Once a time interval is selected and measured, the stability of the measured biometric signal must be determined. For an ECG signal, a stability index (Sx) can be calculated. The stability index (Sx) compares a measured time interval (Tₙ) to the prior measured time interval (Tₙ₋₁) in accordance with the below equation:

$$Sx = \frac{( {T}_{n - 1} )}{( Tn )} \qquad Equation 9$$

The desired value for the stability index (Sx) is one (1). A value of one indicates that the interval before the current cardiac cycle and the prior cardiac cycle are the same. This is indicative of a steady cardiac rhythm and ideal stability. If the stability index (Sx) is less than one (<1), then the cardiac rhythm is increasing. If the stability index is more than one (>1), then the cardiac rhythm is decreasing. The stability index (Sx) is updated after each cardiac cycle, which averages 60 bpm to 90 bpm. Accordingly, the stability index (Sx) is a good dynamic metric of heart rhythm stability.

The measured pulse rhythm and the stability index (Sx) can be used to dynamically control the operation of the cardiac assist device 20. In this manner, the cardiac assist device 20 will synchronize to the rhythm of the heart 11 even as that rhythm is increasing or decreasing. The cardiac assist device 20 will be controlled in a manner that directs the stability index (Sx) to a value of one. Accordingly, if the rhythm of the heart 11 begins to speed up, the cardiac assist device 20 will also speed up, but at a slightly slower rate to keep the stability index (Sx) at one. Likewise, if the rhythm of the heart 11 begins to slow, the cardiac assist device (20) will slow, but at a slightly slower rate to keep the stability index at one.

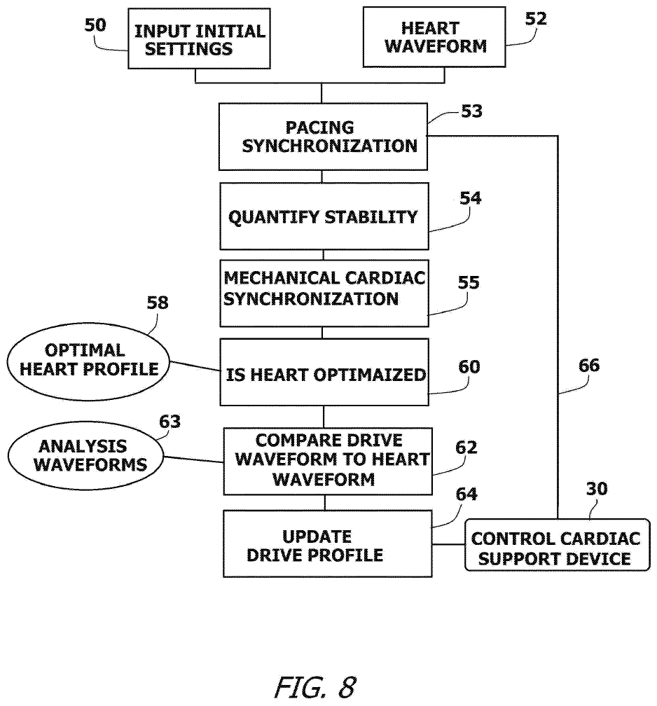

Referring to FIG. 8 in conjunction with FIG. 1, FIG. 2, and FIG. 7, it will be understood that initial settings, such as the make/model of the cardiac assist device 20 and the physiology of the heart 11 are entered. See Block 50. Likewise, the central processing unit 28 reads the biometric signals being used. See Block 52. The biometric signals are obtained from the heart monitoring equipment 16 in use. The central processing unit 28 then calculates pacing synchronization the contraction rate utilized by the cardiac assist device 20 is matched to the currently perceived contraction rate of the heart 11. See Block 53. The contraction rate is determined by the time interval (Tn-1) between the last two cardiac cycles. Pacing synchronization is premised on the understanding that over time, the heart's inherent contraction rate will vary and therefore, re-interrogation is performed after every cardiac cycle to ensure the heart 11 and the cardiac assist device 20 are synchronized. In this manner, the cardiac assist device 20 can improve the heart's pump function while reducing the chance of the cardiac assist device 20 will hinder the heart and put undue pressure on the heart 11 that is not fully functional.

The inherent cardiac cycle rate of the heart 11 varies. That is, the cardiac rhythm is rarely stable but rather increases or decreases across a period of time. In order to quantify the stability of the cardiac rhythm. The stability of the biometric signal is quantified. See Block 54. If the biometric signal is obtained from an ECG, pulse monitor or a like piece of equipment that can directly measure the RR interval, then a stability index (Sx) can be calculated. The stability index (Sx) provides a dynamic indication as to whether the cardiac cycle rate is increasing or decreasing. In this manner, the central processing unit 28 can dynamically adjust the cardiac assist device 20 to both match and control the changing cardiac cycle rate of the heart. The central processing unit 28 generates drive profiles that begins the contraction of the cardiac assist device 20 to maintain the stability index (Sx) at a value of one.

Once pacing synchronization is optimized, the second process of achieving mechanical cardiac synchrony is started. See Block 55. Preferably, in order to achieve mechanical cardiac synchrony, the strain of the heart 11 is considered. One or more physiological parameters for the heart 11, such as size, are input into the central processing unit 28. Accordingly, an optimal strain profile 58 for a heathy heart having such parameters can be mathematically calculated. The central processing unit 28 attempts to achieve mechanical cardiac synchrony. Mechanical cardiac synchrony corresponds to the complex ventricular contractions that occur in the heart 11. The manner in which the ventricles contract can be quantified using strain analysis. Strain analysis can look at all different areas of the heart 11 and determine if contractions follow a relatively ideal contraction “profile”. Mechanical cardiac synchrony for the heart 11 is achieved when the ventricular strain profile measured for the heart 11 matches the optimal strain profile 58. To do this, the central processing unit 28 generates drive profiles 30 for the cardiac assist device 20 so that the forces applied by the cardiac assist device 20 combine with the inherent functionality of the heart 11 to create a modified strain profile that approaches that of the optimal strain profile 58.

The goals of mechanical cardiac synchrony are three-fold. The first goal is to improve the heart's native contractile function, although it may have been severely or partially compromised. The second goal is to improve the likelihood that the compression cycle of the cardiac assist device 20 is synchronized with that of the compression cycle for the heart 11. Lastly, the third goal is to improve the likelihood that that mechanical stimulus of the cardiac assist device 20 leads to the mechanical contraction of the heart 11. When the heart 11 has no intrinsic pulses, the cardiac assist device 20 can, therefore, stimulate the synchronization, and then adjust the level of external support based on how much the heart's native functions are restored.

When heart function is measurable, e.g., greater than approximately 20% of normal function, it is possible to accurately synchronize the cardiac assist device 20 with the heart's native or intrinsic cyclic pump function using the stability index (Sx). As is indicated by Block 60, after mechanical cardiac synchronization is complete, the central processing unit 28 compares the strain profile measured directly from the heart 11 to the optimal strain profile 58. If the measured strain profile is not the same as the optimal strain profile 58, then corrective measures are taken. The drive profiles 30 generated by the central processing unit 28 control the physical operation of the cardiac assist device 20. Accordingly, different drive profiles 30 cause the cardiac assist device 20 to produce different pressure waveforms. The pressure waveforms are estimations produced using the formulations for strain previously described. The differences in strain profiles between the measured strain profiles and the optimal strain profile 58 are known. A drive profile 30 is generated that creates a strain profile that, when added to the measured strain profile, should approach the value of the optimal strain profile. The strain profile generated by the cardiac assist device 20 in response to a drive profile 30 is compared to the heartbeat waveform detected by the heart monitoring equipment 16. See Block 62. Upon comparison of the waveforms, a coordination index (CI) between the two waveforms can be calculated using the following equation.

$$CI = \sqrt{\frac{1}{N − 1} {\sum }_{i = 1}^{N} {( {d}_{i} − \overline{d} )}^{2}} \qquad Eqaution 10$$

The variable “dᵢ” is the time delay between the selected peak heart signals (physiologic waveform) over one device pressure cycle. The variable “d” is the average delay over N number of cycles. The variable “N” is the number of consecutive cycles in recording (e.g., N can be any number greater than 3, but N>10 or any other arbitrary number with increasing N leading is preferred to increase accuracy. The coordination index (CI) is basically the standard deviation of the delay between peaks. The coordination index also represents a very basic mathematical means for demonstrating how one can assess regularity and how an intervention can be made and this used to reassess if the intervention created more regularity. For this reason, it is being used to be exemplary in how one can assess variability.

During the waveform synchronization procedure, it will be understood that the physiological waveform during support with the cardiac assist device 20 is analyzed. The regularity of peaks in the physiological waveform are identified. The physiological waveforms can be complex and must be processed to determine the proper peaks to associate with cardiac rhythm. See Block 63. Fourier analysis is applied to the waveforms to deconstruct the underlying periodic responses. Once peaks are isolated, a coefficient of variation can be derived from the peaks. The coefficient of variation is the standard deviation of the peak's intervals, which is a measure of interval length between peaks.

If it is determined that each physiologic peak is regular, then the device rate is not changed. If the physiological rhythm is irregular the device rate is changed. Analysis is performed over multiple cycles. If higher variability between peak mismatches is seen over repeated cycles, then an updated device profile 30 is created. See Block 64. The device profile 30 can be updated using any combination of three options. The peak time (tₚₑₐₖ) of the cardiac assist device 20 can be adjusted. Second, the mechanical pacing can be adjusted so that the frequencies of contraction better align. Third, the peak strain ε(peak) produced by the cardiac assist device 20 can be adjusted. If one or more of these corrective actions idealizes mechanical cardiac synchrony, then the device specific pressure waveform is achieved. If mechanical cardiac synchrony is not idealized, then the adjustments are repeated. See loop line 66.

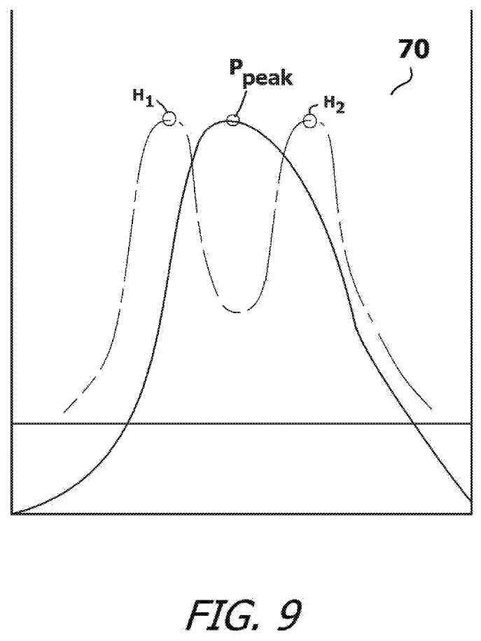

Referring to FIG. 9, a graph 70 is shown that illustrates the identification of the cycles featuring loss of ventricular capture. Loss of ventricular capture occurs when number of peaks from the heartbeat waveform is greater than 1. This is H₁, H₂ and the time between the two adjacent peaks is more than a certain percentage of the length of the device pressure cycle, e.g., >10% of the cycle length with less cycle length indicating more synchrony. It should be understood that the loss of ventricular capture, as represented in FIG. 9, can be detected, and can be corrected in the same manner set forth in the methodology of FIG. 8. That is, the degree of loss can be detected, and an optimal device profile can be generated to minimize the loss in ventricular capture.

It will be understood that the embodiments of the present invention that are illustrated and described are merely exemplary and that a person skilled in the art can make many variations to those embodiments. All such embodiments are intended to be included within the scope of the present invention as defined by the claims.

What is claimed is:

1. A method of supporting functionality of a patient's heart with a cardiac assist device, said method comprising the steps of:
placing said cardiac assist device in position to affect said patient's heart, wherein said cardiac assist device selectively applies mechanical forces to said patient's heart;
monitoring said patient's heart to determine time intervals between cardiac cycles and a rhythm for said time intervals;
determining a stability for said rhythm wherein said stability is indicative of time regularity within said rhythm;
dynamically controlling said cardiac assist device as a function of said stability, wherein said cardiac assist device applies said mechanical forces to said patient's heart to cause said stability to approach regularity.

2. The method according to claim 1, wherein said time intervals include a current time interval and a prior time interval, and wherein said stability is quantified using a stability index, wherein said stability index is obtained by dividing said prior time interval by said current time interval.

3. The method according to claim 1, wherein the cardiac cycles of the patient's heart have a physiological waveform that include physiological peaks.

4. The method according to claim 3, wherein said stability is determined at least in part by comparing differences in regularity between said physiological peaks.

5. The method according to claim 4, further including dynamically adjusting said mechanical forces to reduce differences in regularity between said physiological peaks.

6. The method in claim 5, further including monitoring said pressure peaks to determine if adjusting said mechanical forces has improved regularity between said physiological peaks.

7. The method according to claim 3, wherein comparing said physiological peaks includes calculating a coordination index (CI) using
$$C I = \sqrt{\frac{1}{N − 1} {\sum }_{i = 1}^{N} {( {d}_{i} − \overline{d} )}^{2}}$$
where “dᵢ” is a time interval between a selected one of said physiological peaks and a selected subsequent physiological peak, and wherein “d” is the average delay over N number of cardiac cycles.

8. The method according to claim 1, wherein determining a stability for said rhythm includes evaluating a mathematical consistency for said time regularity within said rhythm.

9. The method according to claim 8, wherein said mathematical consistency is determined using mathematical techniques selected from a group consisting of: time variance, ratio-based spectra, entropy, and temporal patterning.

10. The method according to claim 8, wherein said mathematical consistency is determined using Fourier analysis on said physiological peaks to identify regularity within said physiological peaks.

11. The method according to claim 1, wherein said stability is determined at least in part by comparing differences in regularity between said physiological peaks and said pressure peaks.

12. A method of supporting functionality of a patient's heart with an application of mechanical forces, said method comprising the steps of:
monitoring said patient's heart to determine time intervals between cardiac cycles and a rhythm for said time intervals;
monitoring stability for said rhythm wherein said stability is indicative of time regularity within said rhythm;
dynamically adjusting said mechanical forces as a function of said stability to approach a condition of regularity.

13. The method according to claim 12, wherein said time intervals include a current time interval and a prior time interval, and wherein said stability is quantified using a stability index, wherein said stability index is obtained by dividing said prior time interval by said current time interval.

14. The method according to claim 12, wherein the cardiac cycles of the patient's heart have a physiological waveform that includes physiological peaks.

15. The method according to claim 14, wherein said stability is determined at least in part by comparing differences in regularity between said physiological peaks.

16. The method according to claim 15, further including dynamically adjusting said mechanical forces to reduce differences in regularity between said physiological peaks.

17. The method in claim 12, further including monitoring said pressure peaks to determine if adjusting said mechanical forces has improved regularity between said physiological peaks.

18. The method according to claim 12, wherein determining a stability for said rhythm includes evaluating a mathematical consistency for said time regularity within said rhythm.
